(19) United States
(12) Patent Application Publication
(10) Pub. No.: US 2025/0102431 A1
(43) Pub. Date: Mar. 27, 2025

(54) METHOD FOR DETERMINING FULL CAPSID CONTENT IN ADENOASSOCIATED VIRUSES

(71) Applicant: Access Medical Systems, Ltd., Palo Alto, CA (US)

(72) Inventors: Pu Li, Los Altos, CA (US); Indrani Chakraborty, Fremont, CA (US); Jessie Peh, Saratoga, CA (US)

(21) Appl. No.: 18/972,715

(22) Filed: Dec. 6, 2024

Related U.S. Application Data
(63) Continuation of application No. PCT/US2023/068049, filed on Jun. 7, 2023.
(60) Provisional application No. 63/366,067, filed on Jun. 8, 2022.

(51) Int. Cl. G01N 21/45; G01N 1/44; G01N 33/569

(52) U.S. Cl. CPC G01N 21/45; G01N 1/44; G01N 33/56983; G01N 2333/015

(57) ABSTRACT

The present invention is directed to a method of determining the percentage of full capsids in an adeno-associated virus (AAV) sample comprising AAVs. The invention features two basic steps. The first step involves separating each sample into two aliquots. One aliquot is kept at room temperature (RT), while the other aliquot is heated below the AAV melting point to disrupt the binding affinity of the AAVs to an anti-AAV coated probe. Heating an AAV aliquot leads to differential binding affinity of empty capsids versus full capsids on the anti-AAV-coated probe. The heated empty capsids retain their binding affinity to the probe, while the heated full capsids significantly reduce their binding affinity to the probe. The second step involves measuring the wavelength shifts of heated and RT aliquots due to light interference, and calculating their ratio. By quantitating the ratio against a standard curve, the amount of empty capsids versus full capsids in an unknown sample can be determined.

### CROSS-REFERENCE TO RELATED APPLICATION(S)

This application is a continuation of PCT/US2023/068049, filed Jun. 7, 2023; which claims the benefit of U.S. Provisional Application No. 63/366,067, filed Jun. 8, 2022. The contents of the above-identified applications are incorporated herein by reference in their entireties.

### FIELD OF THE INVENTION

The present invention discloses an immunoassay that is capable of distinguishing empty versus full viral capsids in adeno-associated viruses based on interferometry.

### BACKGROUND OF THE INVENTION

Adeno-associated viruses (AAVs) have emerged as vectors of choice for gene therapy clinical trials because of their long-term expression and lack of pathogenicity in humans. There are twelve serotypes of AAV identified thus far, with AAV2 being the best characterized and most commonly used serotype for gene therapy applications. Wild-type AAV consists of a single-stranded DNA genome (that is up to 4.8 kb in size) that is encapsidated within a proteinaceous shell comprised on 60 viral proteins (VPs). A mixture of VP1, VP2, and VP3 at an approximate molar ratio of 1:1:10, is organized in an icosahedral symmetry to form the viral capsid shell. The VP3 sequence is shared among all the VPs and it is longer than VP2 by 57 amino acids. VP1 is further N-terminally extended compared to VP2, with the VP1 unique (VP1u) region containing a phospholipase A2 (PLA2) domain, required for infectivity.

Differences in amino acid sequence and capsid structure mediate the interaction of the AAV serotypes with different host cell receptors, leading to alternative cell or tissue tropisms. For example, AAV2 has tropism for skeletal muscle, neurons, and vascular smooth muscle cells, whereas AAV8 has tropism for the liver. Another important consequence of the differences in capsid structure between various AAV serotypes, is the stability of the capsid structure to thermal and pH changes. AAV5, for example has the highest melting temperature (Tm) as determined by differential fluorimetry scanning, while AAV2 has the lowest Tm. This unique property has been exploited to distinguish the AAV serotypes from one another.

AAV capsids that contain the therapeutic full-length transgene are referred to as full capsids. The first FDA-approved AAV-related gene therapy is Luxturna, which is an AAV2 vector. An inherent characteristic of the AAV manufacturing process is the production of capsids that are not packaged with the therapeutic transgene; such capsids are referred to as empty capsids. In general, up to 95% of the capsids produced upstream in the cell culture may be empty capsids, and this percentage has been shown to vary significantly between independent vector preparations.

Table 1 shows main types of capsids generated during recombinant AAV production. (Gimpel, et al., Molecular Therapy: Methods & Clinical Development, 20: 740-754, 2021)

TABLE 1

| | Full Capsids | Partially-filled Capsids | Empty Capsids |
|---|---|---|---|
| Causes | Correct genome | Packing of partial vector | No genome |
| | packaging | or host cell DNA | packaging |
| *Ratio of All Capsids | Harvest: <30% | Harvest: <10% | Harvest: >70% |
| | Purified: >70% | Purified: <1% | Purified: <30% |
| Therapeutic Effect | Intended in vivo | Increases risk of | Increases risk of |
| | gene transfer | immunotoxicity, | immunotoxicity, |
| | | reduces transduction | reduces transduction |

*The ratio of all capsids is given at the time of harvest of the rAAV production culture (“Harvest”), e.g., in the cell lysate prior to any purification, and in the product after purification (“Purified”), i.e., after the purification of full capsids from the cell lysate but prior to any polishing steps for the near complete separation of full from empty capsids.

The clinical effect of these empty capsids is not well understood, but it has been suggested that there could be elevated immune responses to high concentrations of viral particles. Empty capsids are unable to provide the intended therapeutic benefit and are therefore considered to be a product-related impurity. Different analytical techniques exist to quantify the amount of empty versus full AAV capsids throughout the manufacturing process and for the final product.

Existing methods are typically low throughput, require either concentrated (10¹²-10¹⁴ viral particles/mL) purified samples or a long assay time. Quantification of AAV titers and determination of AAV empty and full capsids, remain challenging in the development and manufacturing of therapeutic AAV vectors. There is a need for a high-throughput analytical method that can quantify the amounts of empty AAV capsids versus full AAV capsids in a short assay time, and is tolerant of the use of crude samples.

### BRIEF DESCRIPTION OF THE DRAWINGS

FIG. 1A illustrates a biosensor interferometer system. FIG. 1B depicts a biosensor interferometer probe. FIG. 1C depicts another biosensor interferometer probe.

FIG. 2A shows that the first and second reflected light signals form a spectral interference pattern. FIG. 2B shows the spectral interference pattern shifts from T0 to T1 when analyte molecules bind to the analyte-binding molecules on the distal surface of the interference.

FIG. 3 shows melting points of AAV1-AAV10, full capsids and empty capsids in different buffers.

FIG. 4 plots V value (ratio of wavelength shift of heated sample/wavelength shift of room temperature sample) vs % of full AAV2 capsids.

### DETAILED DESCRIPTION OF THE INVENTION

### Definitions

Terms used in the claims and specification are to be construed in accordance with their usual meaning as understood by one skilled in the art except and as defined as set forth below.

“About,” as used herein, refers to within ±10% of the recited value.

An “analyte-binding” molecule, as used herein, refers to any molecule capable of participating in a specific binding reaction with an analyte molecule. Examples include but are not limited to, (i) antigen molecules, for use in detecting the presence of antibodies specific against that antigen; (ii) antibody molecules, for use in detecting the presence of antigens; (iii) protein molecules, for use in detecting the presence of a binding partner for that protein; (iv) ligands, for use in detecting the presence of a binding partner; or (v) single stranded nucleic acid molecules, for detecting the presence of nucleic acid binding molecules.

An “aspect ratio” of a shape refers to the ratio of its longer dimension to its shorter dimension.

A “binding molecule,” refers to a molecule that is capable to bind another molecule of interest.

“A binding pair,” as used herein, refers to two molecules that are attracted to each other and specifically bind to each other. Examples of binding pairs include, but not limited to, an antigen and an antibody against the antigen, a ligand and its receptor, complementary strands of nucleic acids, biotin and avidin, biotin and streptavidin, lectin and carbohydrates. Preferred binding pairs are biotin and streptavidin, biotin and avidin, fluorescein and anti-fluorescein, digioxigenin/anti-digioxigenin.

“An empty AAV” and “a full AAV”, as used herein, refer to the DNA content inside the AAV protein capsid. An empty AAV means the viral capsid contains no DNA. A full AAV means that the viral capsid contains a full-length single stranded DNA genome. A X % full AAV sample means that X % of the capsids have full-length single stranded DNA genome. For example, 60% full AAV sample means that 60% of the capsids have full-length single stranded DNA genome, and 40% capsids have no full-length single stranded DNA genome.

“Immobilized,” as used herein, refers to reagents being fixed to a solid surface. When a reagent is immobilized to a solid surface, it is either be non-covalently bound or covalently bound to the surface.

“A monolithic substrate,” as used herein, refers to a single piece of a solid material such as glass, quartz, or plastic that has one refractive index.

A “probe,” as used herein, refers to a monolithic substrate having as aspect ratio (length-to-width) of at least 2 to 1 with a thin-film layer coated on the sensing side. A probe has a distal end and a proximal end. The proximal end (also refers to probe tip in the application) has a sensing surface coated with a thin layer of analyte-binding molecules.

A “waveguide” refers to a device (e.g., a duct, coaxial cable, or optic fiber) designed to confine and direct the propagation of electromagnetic waves (as light).

The present invention provides a method of determining the percentage of full capsids of adeno-associated viruses (AAVs) in a sample comprising AAVs. The invention features two basic steps. The first step involves separating each sample into two aliquots. One aliquot is kept at room temperature (RT), while the other aliquot is heated to disrupt the binding affinity of the AAVs to an anti-AAV coated probe. Without heating, there is little difference in the binding affinity of empty AAV capsids or full AAV capsids on the anti-AAV coated glass biosensor probe. However, heating the AAV aliquot leads to differential binding affinity of empty capsids versus full capsids on the anti-AAV-coated probe. The heated empty capsids retain their binding affinity to the probe, while the heated full capsids significantly reduce their binding affinity to the probe. The second step involves measuring the wavelength shifts of heated and RT aliquots due to light interference, and calculating their ratio. By quantitating the ratio against a standard curve, the amount of empty capsids versus full capsids in an unknown sample can be determined.

### Biosensor Interferometer Systems

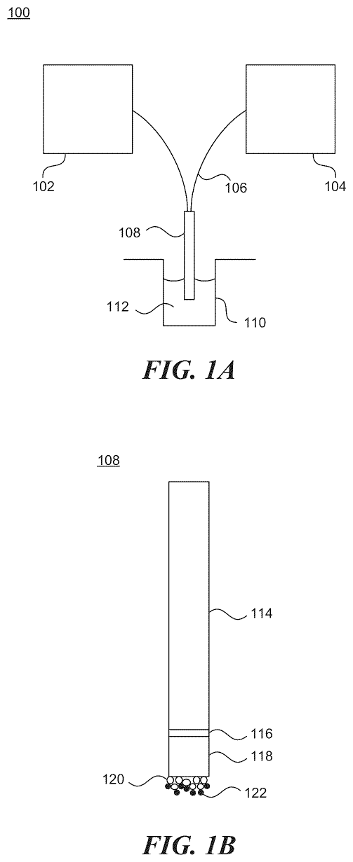

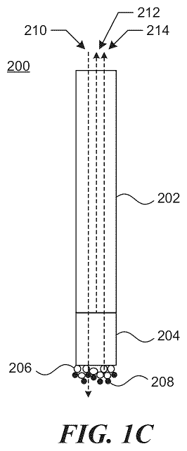

The present invention is suitable to be used with biosensor interferometer systems. FIGS. 1A-B illustrate one example of such a system. FIG. 1A depicts a biosensor interferometer 100 (or simply “interferometer”) that includes a light source 102, a detector 104, a waveguide 106, and an optical assembly 108 (also referred to as a “probe”). The probe 108 may be connected to the waveguide 106 via a coupling medium.

The light source 102 may emit white light that is guided toward the probe 108 by the waveguide 106. For example, the light source 102 may be a light-emitting diode (LED) that is configured to produce light over a range of at least 50 nanometers (nm), 100 nm, or 150 nm within a given spectrum (e.g., 400 nm or less to 700 nm or greater). Alternatively, the interferometer 100 may employ a plurality of light sources having different characteristic wavelengths, such as LEDs designed to emit light at different wavelengths in the visible range. The same function could be achieved by a single light source with suitable filters for directing light with different wavelengths onto the probe 108.

The detector 104 is preferably a spectrometer, such as an Ocean Optics USB4000, that is capable of recording the spectrum of interfering light received from the probe 108. Alternatively, if the light source 102 operates to direct different wavelengths onto the probe 108, then the detector 104 can be a simple photodetector capable of recording intensity at each wavelength. In another embodiment, the detector 104 can include multiple filters that permit detection of intensity at each of multiple wavelengths.

The waveguide 106 can be configured to transport light emitted by the light source 102 to the probe 108, and then transport light reflected by surfaces within the probe 108 to the detector 104. In some embodiments the waveguide 106 is a bundle of optical fibers (e.g., single-mode fiber optic cables), while in other embodiments the waveguide 106 is a multi-mode fiber optic cable.

As shown in FIG. 1B, the probe 108 includes a monolithic substrate 114, a thin-film layer (also referred to as an “interference layer”), and a biomolecular layer (also referred to as a “biolayer”) comprised of analyte molecules 122 that have bound to analyte-binding molecules 120. The monolithic substrate 114 is comprised of a transparent material through which light can travel. The interference layer is also comprised of a transparent material. When light is shone on the probe 108, the proximal surface of the interference layer may act as a first reflecting surface and the biolayer may act as a second reflecting surface. As further described below, light reflected by the first and second reflecting surfaces may form an interference pattern that can be monitored by the interferometer 100.

The interference layer normally includes multiple layers that are combined in such a manner to improve the detectability of the interference pattern. For example, the interference layer is comprised of a tantalum pentoxide (Ta₂O₅) layer 116 and a silicon dioxide (SiO2) layer 118. The tantalum pentoxide layer 116 may be thin (e.g., on the order of 10-40 nm) since its main purpose is to improve reflectivity at the proximal surface of the interference layer. Meanwhile, the silicon dioxide layer 118 may be comparatively thick (e.g., on the order of 650-900 nm) since its main purpose is to increase the distance between the first and second reflecting surfaces.

To illustrate a simple interferometry test, the probe 108 can be suspended in a well 110 that includes a sample 112. Analyte molecules 122 will bind to the analyte-binding molecules 120 along the distal end of the probe 108 over the course of the diagnostic test, and these binding events will result in an interference pattern that can be observed by the detector 104. The interferometer 100 can monitor the thickness of the biolayer formed along the distal end of the probe 108 by detecting shifts in a phase characteristic of the interference pattern.

FIG. 1C illustrates another biosensor interferometer probe. The probe includes a monolithic substrate that has a first and a second surfaces arranged substantially parallel to one another at opposite ends of the monolithic substrate, an interference layer coated on the second surface of the monolithic substrate, and a layer of analyte-binding molecules coated on the interference layer. The interference layer will generally be comprised of magnesium fluoride (MgF₂). A first interface between the monolithic substrate and the interference layer acts as a first reflecting surface when light is shone on the interferometric sensor, while a second interface between a biolayer formed by analyte molecules in a sample binding to the analyte-binding molecules and a solution containing the sample acts as a second reflecting surface when the light is shone on the probe. As described above, the thickness of the biolayer can be estimated based on the interference pattern of light reflected by the first and second reflecting surfaces.

The probe 200 includes an interference layer 204 that is secured along the distal end of a monolithic substrate 202. Analyte-binding molecules 206 can be deposited along the distal surface of the interference layer 204. Over the course of a biochemical test, a biolayer will form as analyte molecules 208 in a sample bind to the analyte-binding molecules 206.

As shown in FIG. 1C, the monolithic substrate 202 has a proximal surface (also referred to as a “coupling side”) that can be coupled to, for example, a waveguide of an interferometer and a distal surface (also referred to as a “sensing side”) on which additional layers are deposited. Generally, the monolithic substrate 202 has a length of at least 3 millimeters (mm), 5 mm, 10 mm, or 15 mm. In a preferred embodiment, the aspect ratio (length-to-width) of the monolithic substrate 202 is at least 5 to 1. In such embodiments, the monolithic substrate 202 may be said to have a columnar form. The cross section of the monolithic substrate 202 may a circle, oval, square, rectangle, triangle, pentagon, etc. The monolithic substrate 202 preferably has a refractive index that is substantially higher than the refractive index of the interference layer 204, such that the proximal surface of the interference layer 204 effectively reflects light directed onto the probe 200. The preferred refractive index of the monolithic substrate may be higher than 1.5, 1.8, or 2.0. Accordingly, the monolithic substrate 202 may be comprised of a high-refractive-index material such as glass (refractive index of 2.0) rather than a low-refractive-index material such as quartz (refractive index of 1.46) or plastic (refractive index of 1.32-1.49).

The interference layer 204 is comprised of at least one transparent material that is coated on the distal surface of the monolithic substrate 202. These transparent material(s) are deposited on the distal surface of the monolithic substrate 202 in the form of thin films ranging in thickness from fractions of a nanometer (e.g., a monolayer) to several micrometers. The interference layer 204 may have a thickness of at least 500 nm, 700 nm, or 900 nm. An exemplary thickness is between 500-5,000 nm (and preferably 800-1,200 nm). Here, for example, the interference layer 204 has a thickness of approximately 900-1,000 nm, or 940 nm.

In contrast to conventional probes, the interference layer 204 has a substantially similar refractive index as the biolayer. This ensures that the reflection from the distal end of the probe 200 is predominantly due to the analyte molecules 208 rather than the interface between the interference layer 204 and the analyte-binding molecules 206. In some embodiments, the interference layer 204 is comprised of magnesium fluoride (MgF₂), while in other embodiments the interference layer 204 is comprised of potassium fluoride (KF), lithium fluoride (LiF), sodium fluoride (NaF), lithium calcium aluminum fluoride (LiCaAlF₆), sodium aluminum fluoride (Na3AlF₆), strontium fluoride (SrF₂), aluminum fluoride (AlF₃), sulphur hexafluoride (SF₆), etc. Magnesium fluoride has a refractive index of 1.38, which is substantially identical to the refractive index of the biolayer formed along the distal end of the probe 200. For comparison, the interference layer of conventional probes is normally comprised of silicon dioxide, and the refractive index of silicon dioxide is approximately 1.4-1.5 in the visible range. Because the interference layer 204 and biolayer have similar refractive indexes, light will experience minimal scattering as it travels from the interference layer 204 into the biolayer and then returns from the biolayer into the interference layer 204.

In one embodiment, the probe 200 includes an adhesion layer that is deposited along the distal surface of the interference layer 204 affixed to the monolithic substrate 202. The adhesion layer may be comprised of a material that promotes adhesion of the analyte-binding molecules 206. One example of such a material is silicon dioxide. The adhesion layer is generally very thin in comparison to the interference layer 204, so its impact on light traveling toward, or returning from, the biolayer will be minimal. For example, the adhesion layer 310 may have a thickness of approximately 3-10 nm, while the interference layer 304 may have a thickness of approximately 800-1,000 nm. The biolayer formed by the analyte-binding molecules 306 and analyte molecules 308 will normally have a thickness of several nm.

When light is shone on the probe 200, the proximal surface of the interference layer 204 may act as a first reflecting surface and the distal surface of the biolayer may act as a second reflecting surface. The presence, concentration, or binding rate of analyte molecules 208 to the probe 200 can be estimated based on the interference of beams of light reflected by these two reflecting surfaces. As analyte molecules 208 attach to (or detach from) the analyte-binding molecules 206, the distance between the first and second reflecting surfaces will change. Because the dimensions of all other components in the probe 200 remain the same, the interference pattern formed by the light reflected by the first and second reflecting surfaces is phase shifted in accordance with changes in biolayer thickness due to binding events.

In operation, an incident light signal 210 emitted by a light source is transported through the monolithic substrate 202 toward the biolayer. Within the probe 200, light will be reflected at the first reflecting surface resulting in a first reflected light signal 212. Light will also be reflected at the second reflecting surface resulting in a second reflected light signal 214. The second reflecting surface initially corresponds to the interface between the analyte-binding molecules 206 and the sample in which the probe 200 is immersed. As binding occurs during the biochemical test, the second reflecting surface becomes the interface between the analyte molecules 208 and the sample.

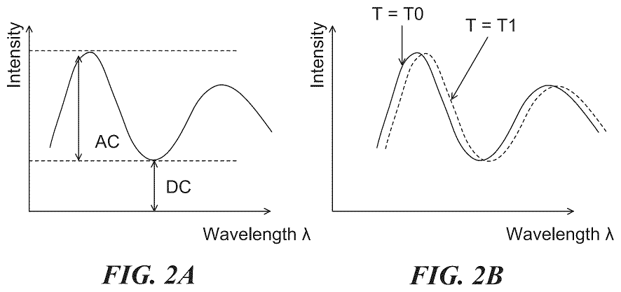

The first and second reflected light signals form a spectral interference pattern, as shown in FIG. 2A. When analyte molecules bind to the analyte-binding molecules on the distal surface of the interference layer, the optical path of the second reflected light signal will lengthen. As a result, the spectral interference pattern shifts from T0 to T1 as shown in FIG. 2B. By measuring the phase shift after a period of time, the association of an analyte molecule to an analyte-binding molecule immobilized on the distal surface of the interference layer 204 can be used to calculate analyte concentration in the sample. By measuring the phase shift continuously in real time, a kinetic binding curve can be plotted as the amount of shift versus the time. The association rate of an analyte molecule to an analyte-binding molecule immobilized on the distal surface of the interference layer can be used to calculate analyte concentration in the sample. Hence, the measure of the phase shift is the detection principle of a thin-film interferometer.

### Immunoassay

The present invention is directed to a method of determining the percentage of full capsids of adeno-associated viruses (AAVs) in a sample comprising AAVs. The method comprises the steps of: (a) obtaining a first probe and a second probe having the same fixed amount of the same anti-AAV antibody immobilized on the tip of the probe; (b) heating an aliquot of the sample at 2-18° C. below the melting temperature of the AAVs; (c) dipping the first probe tip in the heated aliquot of (b) for a first period of time to capture AAVs on the probe in a defined binding condition and measuring the first wavelength shift due to light interference; (d) dipping the second probe tip in a non-heated aliquot from the sample for a second period of time to capture AAVs on the second probe in the same defined binding condition as in step (c), and measuring the second wavelength shift for a second period of time due to light interference; (e) determining the ratio of the first wavelength shift vs. the second wavelength, and quantitating the ratio against a calibration curve to determine the percentage of full capsids of AAVs in the sample.

In step (a) of the present method, two probes that have a small tip for binding an analyte are obtained. The two probes have the same fixed amount of the same anti-AAV antibody immobilized on the tip of the probe such that they have the same ability and capacity to capture AAVs.

In one embodiment, the tip has a smaller surface area with a diameter ≤5 mm, preferably ≤2 mm or ≤1 mm. A probe having a small surface area is advantageous because it has less non-specific binding and thus produces a lower background signal

Methods to immobilize a protein such as an antibody to a solid phase (the sensing surface of the probe tip) are known in immunochemistry. A protein can bind directly to the solid phase through adsorption or it can bind indirectly to the solid phase through a binding pair. For example, the probe surface can be coated with a first member of the binding pair (e.g. anti-hapten), and an anti-AAV antibody labelled with a second member of the binding pair (e.g., hapten) is immobilized on the probe through the biotin-streptavidin binding.

In step (b) of the method, an aliquot of the sample is removed from a sample and is heated at about 2-18° C. below the melting temperature of the AAVs from about 2 minutes up to 3 hours.

The inventors have discovered that heating full AAV capsids results in a significant reduction in the binding affinity of full AAV capsids to the anti-AAV coated probes. On the contrary, heating empty AAV capsids does not results in a significant reduction in the binding affinity of empty AAV capsids to the anti-AAV coated probes. In general, heating full AAV2 capsids at 50-65° C. in PBS buffer for 30 minutes results in decreasing the binding affinity of full AAV capsids to the anti-AAV coated probes to 15-35%, comparing with that of non-heated full AAV2 capsids. In general, heating empty AAV2 capsids at 50-65° C. in PBS buffer for 30 minutes results in changing the binding affinity of empty AAV capsids to the anti-AAV coated probes to 75-90%, relative to the unheated sample.

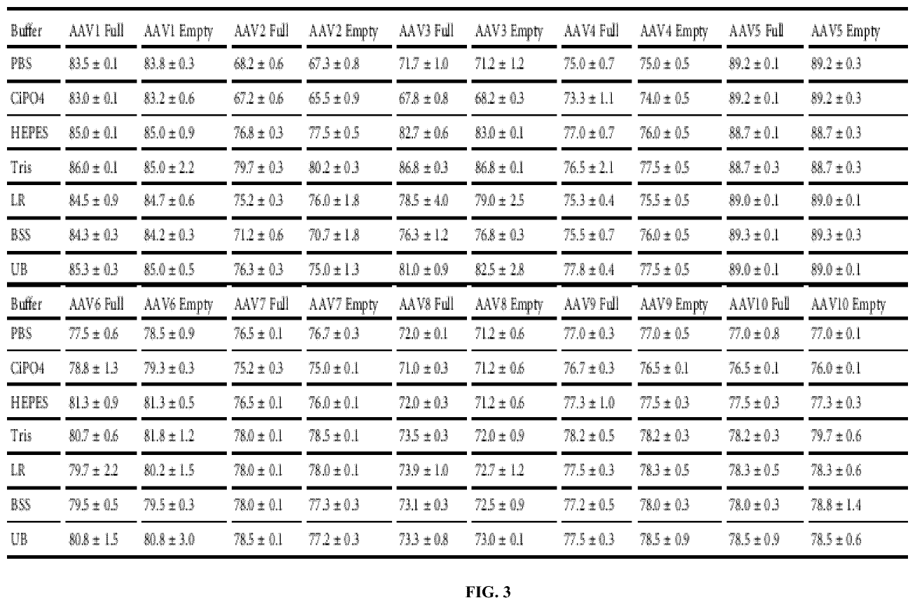

The heating is performed at about 2-18° C., or about 5-12° C. below the melting temperature of AAVs. The melting points of AAV1-AAV9 and AAVrh.10, full and empty capsids, in different buffers, are shown in FIG. 3. (Bennett, et al. Molecular Therapy: Methods & Clinical Development, 6: 171-182 (2017))

For example, when an AAV sample is in PBS buffer, the heating temperature range is about 65-80° C. for AAV1, about 50-65° C. for AAV2, about 55-70° C. for AAV3, about 55-72° C. for AAV4, about 70-85° C. for AAV5, about 60-75° C. for AAV6, about 60-75° C. for AAV7, about 55-70° C. for AAV8, about 60-75° C. for AAV9, and about 60-75° C. for AAV10.

The AAV aliquot is heated to change the binding affinity of AAV full capsids to anti-AAV immobilized on the probe. The heating time is from 2-5 minutes to 1-3 hours, for example, 2 minutes to 1 hour, 5 minutes to 2 hours, 5 minutes to 1 hour, 10 minutes to 1 hour, or 10 minutes to 30 minutes, or 20 minutes to 40 minutes, depending on the AAV type and AAV concentration.

In step (c), the first probe tip is dipped in the heated aliquot of (b) in a defined binding condition for a first period of time to capture AAVs on the probe and the first wavelength shift due to light interference is measured.

In step (d), a second probe tip is dipped in a non-heated aliquot from the same sample for a second period of time to capture AAVs on the second probe in the same defined binding condition as in step (c), and the second wavelength shift due to light interference is measured.

The non-heated aliquot stays at room temperature (e.g., 18-40° C.) until ready for testing.

The binding conditions in steps (c) and (d) are the same, and the first and second period of time for binding in steps (c) and (d) are the same; and therefore, the difference in amounts of AAV capsids bound to the anti-AAV coated probes in heated and non-heated aliquots, if any, are solely due to heating vs. non-heating procedures.

The wavelength shifts in steps (c) and (d) can be measured by kinetic protocol or endpoint protocol. Kinetic measurement refers to measuring change of wavelength shift vs. time, typically in nanometer wavelength shift per second. Endpoint measurement refers to measuring wavelength change after a period of time, e.g., nanometer wavelength shift after 2-60 minutes, or 5-40 minutes.

In step (e), the ratio of the first wavelength shift vs. the second wavelength shift is determined. The ratio is then quantitated against a pre-established calibration curve having wavelength shifts plotted against percentages of full capsids of AAVs in the sample, to determine the percentage of full capsids of AAVs in the sample. The calibration curve was pre-established under the same heating and binding conditions with probes coated with the same amounts of same anti-AAV antibody.

The invention is an easy and accurate method to determine the percentage of full capsids of adeno-associated viruses (AAVs) in a sample comprising AAVs.

The invention is illustrated further by the following examples that are not to be construed as limiting the invention in scope to the specific procedures described in them.

### EXAMPLES

### Instrumentation

GatorPrime and GatorPro systems (Gator Bio, Inc.) were used for BLI measurement. ProFlex PCR system (Invitrogen) was used to heat the samples up to 60° C. for 30 min.

### Example 1. Preparation of Reagents and Anti-AAV2 Probe for the Immunoassay

Quartz probes, 1 mm diameter and 2 cm in length, with BLI optical layer at the distal tip were coated with aminopropylsilane using a chemical vapor deposition process (Yield Engineering Systems, 1224P) following manufacturer's protocol. FIGS. 1B and 1C show BLI probes. The probe tip was then functionalized with 1 mg/mL bis(sulfosuccinimidyl)suberate (ThermoFisher Scientific 21580) in 10 mM sodium phosphate buffer pH 3.8 for 2 minutes. This was followed by the immobilization of native Streptavidin (IBA) at 50 μg/mL in 10 mM sodium phosphate buffer pH 3.8 for 10 minutes. Finally, biotinylated anti-AAV2 antibody (Genscript A02160) diluted to 10 μg/mL in PBS pH 7.4 buffer (Sigma-Aldrich P3813) was immobilized on the probe for 10 minutes. Probes were washed with PBS then immersed in a solution of 15% sucrose followed by drying in an oven at 40° C. for 20 minutes.

### Example 2. Wavelength Shift Ratio of Room Temperature Sample and Heated Sample

0% full (empty) or 100% full (full) AAV2 capsids (Sirion Biotech) were diluted to 9.0×10¹⁰ vp/mL, 3.0×10¹⁰ vp/mL, and 1.0×10¹⁰ vp/mL in Q buffer (PBS with 0.2% BSA, 0.02% Tween-20, and 0.02% Proclin-30). Half of the diluted capsids were kept at room temperature (RT) and the other half were heated at 60° C. for 30 minutes.

Then, 100 μL of each RT sample and each heated sample were pipetted into a 384-well black plate (Greiner) with anti-AAV2 coated probe inserted and ran on the GatorPlus instrument to determine their wavelength shift according to the protocol of Table 2 below.

TABLE 2

| Step | | | Time | Speed |
|---|---|---|---|---|
| Number | Reaction | Sample | (Sec) | (RPM) |
| 1 | Baseline run | Q buffer | 60 | 1000 |
| 2 | AAV2 | AAV2 capsids | 1800 | 1000 |
| | binding | in Q buffer | | |

At the end of run, each nanometer (NM) shift was determined and the V value (ratio of NM shift at 60° C./NM shift at RT) was calculated. The results are shown in Table 3.

TABLE 3

| AAV | Ratio of NM shift | Ratio of NM shift |
|---|---|---|
| Concentration | 60° C./RT | 60° C./RT |
| (vp/mL) | Empty | Full |
| 9.0 × 10¹⁰ | 1.01 | 0.38 |
| 3.0 × 10¹⁰ | 1.02 | 0.31 |
| 1.0 × 10¹⁰ | 1.07 | 0.35 |

The results of Table 3 show that using 9.0 E10, 3.0 E10, and 1.0 E10 vp/mL of capsids, the value of V for empty capsids remains relatively constant at around 1 (1.01-1.07). For full capsids, the values of V also remain relatively constant (0.31-0.38), regardless of the input capsid concentration.

### Example 3. Standard Curve and Test Sample

Standards of AAV2 samples with 0%, 15%, 30%, 45%, 60%, 75%, 100% of full capsids were prepared by mixing various amounts of 0% full (empty) and 100% full (full) capsids. The test sample in this example was prepared by mixing equal amounts of 0% full (empty) and 100% full (full) capsids to yield a 50% full sample.

AAV2 capsids (Sirion Biotech) were diluted to 1.8 E10 vp/mL in Q buffer (PBS with 0.2% BSA, 0.02% Tween-20, and 0.02% Proclin-30). For each standard and test sample, half of the sample was kept at room temperature and the other half was heated at 60° C. for 30 minutes. Then, 100 μL of each RT sample and each heated sample were pipetted into a 384-well black plate (Greiner) with anti-AAV2 coated probe inserted and ran on the GatorPlus instrument to determine their wavelength shift according to the same protocol of Table 1.

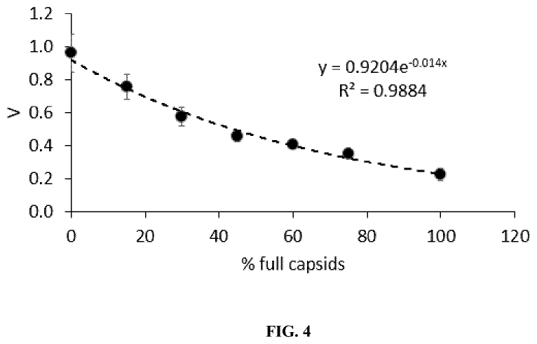

At the end of run, each nanometer (NM) shift was determined and the V value (ratio of NM shift at 60° C./NM shift at RT) was calculated. The results are shown in Table 4 and are plotted to generate a standard curve as shown in FIG. 4. The % CV is less than 20% for all the percentage capsid contenta (0%, 15%, 30%, 45%, 60%, 75%, and 100% of full) tested.

TABLE 4
V value

| % Full | Rep 1 | Rep 2 | Rep 3 | Ave | CV |
|---|---|---|---|---|---|
| 0 | 0.967 | 0.842 | 1.07 | 0.961 | 12% |
| 15 | 0.822 | 0.677 | 0.78 | 0.759 | 10% |
| 30 | 0.636 | 0.529 | 0.56 | 0.574 | 10% |
| 45 | 0.486 | 0.441 | 0.44 | 0.455 | 6% |
| 60 | 0.433 | 0.403 | 0.39 | 0.408 | 6% |
| 75 | 0.373 | 0.329 | 0.35 | 0.349 | 6% |
| 100 | 0.213 | 0.266 | 0.20 | 0.225 | 16% |

The V value of the test sample (50% full) is quantitated against the standard curve (FIG. 4) to determine its concentration. The results are shown in Table 5.

TABLE 5
Quantitation of the Test Sample

| | Rep 1 | Rep 2 | Rep 3 | Ave |
|---|---|---|---|---|
| Actual (% Full) | 50.0 | 50.0 | 50.0 | 50.0 |
| Calculated (% Full) | 52.1 | 42.0 | 64.6 | 52.9 |
| Difference (% Full) | 2.1 | 8.0 | 14.6 | 8.2 |

Table 5 shows that the average difference of the calculated capsid content from the known capsid content is 8.2% over 3 independent experiments.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

What is claimed is:

1. A method of determining the percentage of full capsids of adeno-associated viruses (AAVs) in a sample comprising AAVs, comprising the steps of:
(a) obtaining a first probe and a second probe having the same fixed amount of the same anti-AAV antibody immobilized on the tip of the probe;
(b) heating an aliquot of the sample at 2-18° C. below the melting temperature of the AAVs;
(c) dipping the first probe tip in the heated aliquot of (b) for a period of time to capture AAVs on the probe in a binding condition and measuring the first wavelength shift due to light interference;
(d) dipping the second probe tip in a non-heated aliquot from the sample for the same period of time to capture AAVs on the second probe in the same binding condition as in step (c), and measuring the second wavelength shift for a second period of time due to light interference; and
(e) determining the ratio of the first wavelength shift vs. the second wavelength, and quantitating the ratio against a calibration curve to determine the percentage of full capsids of AAVs in the sample.

2. The method of claim 1, wherein the AAV is AAV2.

3. The method of claim 2, wherein the aliquot of (b) is heated at 50-65° C. for 5 minutes to an hour.

4. The method of claim 1, wherein the AAV is AAV1, and the aliquot of (b) is heated at 65-80° C.

5. The method of claim 1, wherein the AAV is AAV3, and the aliquot of (b) is heated at 55-70° C.

6. The method of claim 1, wherein the AAV is AAV4, and the aliquot of (b) is heated at 55-72° C.

7. The method of claim 1, wherein the AAV is AAV5, and the aliquot of (b) is heated at 70-85° C.

8. The method of claim 1, wherein the AAV is AAV6, and the aliquot of (b) is heated at 60-75° C.

9. The method of claim 1, wherein the AAV is AAV7, and the aliquot of (b) is heated at 60-75° C.

10. The method of claim 1, wherein the AAV is AAV8, and the aliquot of (b) is heated at 55-70° C.

11. The method of claim 1, wherein the AAV is AAV9, and the aliquot of (b) is heated at 60-75° C.

12. The method of claim 1, wherein the AAV is AAV10, and the aliquot of (b) is heated at 60-75° C.
